(19) United States
(12) Patent Application Publication
(10) Pub. No.: US 2010/0227042 A1
(43) Pub. Date: Sep. 9, 2010

(54) Enzyme-Assisted De-Emulsification of Aqueous Lipid Extracts

(76) Inventors: Christopher Penet, Manakin Sabot, VA (US); Peter Birschbach, Rochester, NY (US); Buddhi P. Lamsal, Manhattan, KS (US); Lawrence A. Johnson, Ames, IA (US); Stephanie E. Glatz, Ames, IA (US); Charles E. Glatz, Ames, IA (US); Cheng Zhang, Ames, IA (US); Jianping Wu, Edmonton (CA)

Correspondence Address: DANISCO US INC.;ATTENTION: LEGAL DEPARTMENT, 925 PAGE MILL ROAD, PALO ALTO, CA, 94304, US

(21) Appl. No.: 12/520,028

(22) PCT Filed: Dec. 4, 2007
(86) PCT No.: PCT/US2007/024897

§ 371 Date: Apr. 14, 2010

Related U.S. Application Data
(60) Provisional application No. 60/876,879, filed on Dec. 22, 2006.

(51) Int. Cl. A23L 1/28; B01D 17/04; C11B 1/00; A23L 1/305; C07K 2/00

(52) U.S. Cl. 426/655; 435/266; 554/115; 530/350; 426/656

(57) ABSTRACT

Compositions and processes for destabilizing an oil-in-water emulsion resulting from the aqueous solvent extraction of plant oils are disclosed. The processes comprise the use of one or more enzyme activities including phospholipase and protease activity. The processes are useful for improving the extraction of oil from oilseeds, as well as for obtaining more desirable proteins from those oilseeds.

### FIELD OF THE INVENTION

This pertains to the use of enzymes to aid in the extraction of lipids from plants or plant tissues. More particularly, it pertains to compositions comprising enzymes for de-emulsifying emulsions obtained from aqueous extraction of lipids from plants, and improved methods for their use.

### BACKGROUND

Plant-derived lipids, particularly oils, are a major source of lipids for food processing, and for industrial feedstock. More recently they are of interest and use as alternatives to petrochemicals for fuels. Plant lipids can be derived from one or more parts of a plant, shrub, or tree. In various plants, the root, stem, bark, leaves, flowers, seeds, fruits, or other parts may serve as a source of oil. Such lipids can be extracted mechanically, e.g. through the application of external pressure, or chemically, e.g. through organic or aqueous solvent extraction processes, or combination processes.

Plant oils are often classified as either essential or fixed. Essential oils are volatile oils typically derived from tissues other than the seed of a plant. Fixed oils, also called “fatty oils” include oils derived from plants sources, for example, soybean, corn, canola (rapeseed), sunflower, safflower, peanut, coconut, copra, palm, cottonseed, olive, sesame, flaxseed, and others.

Oils obtained from oilseed plants are known for their uses in food, and food products, as well as for soaps, detergents, lotions, lubricants, insecticides, paints, coatings, inks, and other industrial or consumer products. Although the vast majority of oilseeds oils are extracted with an organic solvent extraction process, some are now extracted through aqueous extraction.

Extraction with organic solvents is faster and more efficient, providing higher yields of oil. This is by far the dominant commercial process for extraction of, for example, soybean oil, with greater than 90% of all soy bean oil extracted with hexane or other organic solvents. However, such solvents pose substantial safety issues, including the risk of fire and explosions from solvents, as well as exposure risks to personnel. Solvent extraction processes require expensive plants that are equipped to handle the solvents, as well as the required the safety measures. In addition, the large-scale use of organic solvents raises waste management concerns. Furthermore, for applications in food processing, the solvent-based extractions may result in denatured, altered, or less functional proteins in the remaining, extracted portion of the oilseed (e.g. the “meal”), decreasing the value or use of this potentially more valuable portion of the oilseed.

Aqueous processes for extracting oils from oilseeds have been developed in recent years. See Freitas et al., Fett/Lipid 99: 333-337, 1997; and Caetano et al., La Rivista Italiana Delle Sostanze Grasse 79: 165-169, 2002. Both Freitas et al. and Caetano et al. provide a combination of extrusion and protease treatment to extract oils in aqueous processes. Aqueous process steps are inherently safer than organic solvent extraction steps and accordingly, the initial start-up investment in capital equipment is substantially less for aqueous extraction processes. Aqueous extraction processes, however, frequently result in less efficiency and decreased yields of oils as compared to organic solvent extraction.

There is a need therefore in the art for processing steps to improve aqueous extraction methods for obtaining oils from plant sources, especially oilseeds. There is also a need to increase the efficiency or yields of such processes.

### SUMMARY

In several of its various aspects, processes are provided for using de-emulsifying oil-in-water emulsions obtained from aqueous extraction of plant tissues, particularly oilseeds, for example by the use of various enzymes. Also provided are methods for obtaining oil from oilseeds wherein the method includes aqueous extraction and enzymatic de-emulsification of a resulting oil-in-water emulsion. Oil derived by the processes and methods, as well as compositions comprising the oil such as food, consumer products and industrial feedstocks are also provided. Compositions comprising enzymes capable of de-emulsifying an oil-in-water emulsion and further comprising an oil-in-water emulsion are also provided herein.

In one aspect, provided are processes for destabilizing an emulsion comprising an oil phase and an aqueous phase, where the emulsion has been produced in an aqueous solvent extraction of a lipid from an oilseed. The process comprises the step of contacting the emulsion with at least one enzyme activity including at least a phospholipase or a protease, or a combination thereof, under conditions allowing activity of at least the phospholipase or the protease, or the combination, for a time sufficient to destabilize the emulsion. In certain preferred embodiments the water phase is the continuous phase of the emulsion and the oil phase is the discontinuous phase of the emulsion.

In another aspect, processes are provided for obtaining oil from an oilseed. The processes generally comprise the steps of:
(a) providing an oil-containing oilseed fraction;
(b) contacting the oil-containing oilseed fraction with an aqueous extractant to form an extracted oilseed fraction;
(c) separating the extracted oilseed fraction into an aqueous phase, an oil-in-water emulsion, and an insoluble phase;
(d) contacting the oil-in-water emulsion with at least one enzyme activity under conditions permitting enzyme activity for a time sufficient to destabilize the emulsion; and
(e) separating the destabilized emulsion into an aqueous phase, an oil phase, and an insoluble phase.

The processes result in obtaining oil from the oilseed. In one embodiment, the oil-containing oilseed fraction comprises cells and the process further comprises the step of disrupting the cells prior to contacting the oilseed fraction with the aqueous extractant.

Also provided herein are plant-derived oils prepared by the above disclosed processes, as well as a host of food products comprising the oil so obtained.

In another aspect of the invention, compositions are provided comprising at least one enzyme activity capable of de-stabilizing an oil-in water emulsion; and an oil-in-water emulsion obtained from an aqueous solvent extraction of an oil-containing oilseed fraction. IN one embodiment the enzyme activity comprises at least a phospholipase or a protease, or any combination of one or more such activities. Also provided herein are plant-derived oils isolated from the compositions taught herein.

Another aspect provides methods for obtaining plant oil from an oilseed fraction. The methods comprise the steps of:
(a) providing a composition comprising at least one enzyme activity capable of de-stabilizing an oil-in water emulsion, and an oil-in-water emulsion obtained from an aqueous solvent extraction of an oil-containing oilseed fraction;
(b) providing conditions under which the enzyme activity de-stabilizes the oil-in-water emulsion; and
(c) separating the composition into at least an aqueous phase and a lipid phase, where the lipid phase comprises the plant oil.

Also provided herein are plant-derived oil comprising plant oil prepared by the methods disclosed, as well as food products, consumer products, and industrial feedstocks comprising plant oil so prepared or obtained. Protein compositions prepared in accordance with the methods provided herein are also provided—these are useful as sources of protein of improved functionality. Food products, consumer products, and industrial feedstocks comprising protein prepared by the methods taught herein are also provided.

Bio-fuels or eco-fuels comprising the plant oils or proteins prepared by any of the methods disclosed are also provided herein.

These and other aspects will be further illustrated through the following detailed description, figures, and examples, which are intended to illustrate various embodiments.

### BRIEF DESCRIPTION OF THE DRAWINGS

The appended figures serve to further explain or illustrate various aspects of the compositions or methods described more fully herein and exemplified below.

FIG. 1: Yield of oil recovered as a function of enzyme concentration. Filled circles: Fungal Protease Concentrate; Open squares: LysoMax® enzyme. Reactions incubated for 90 min at 50° C.

FIG. 2: Yield of oil recovered, as a function of pH. The pH of the cream before adjustment was 8.0.

FIG. 3: Flow chart showing an exemplary aqueous extraction process for obtaining oil from oilseed.

FIG. 4: Oil distribution in specified fractions after enzyme-assisted aqueous extraction, and o/w emulsion breakdown after aqueous extraction plus and enzyme-assisted de-emulsification with treatments as indicated. Symbols are as follows: squares with: hash marks=control; diagonal stripes=phospholipase C; diamonds=Genencor enzyme cocktail; horizontal stripes=freeze-thaw; solid squares=heat treatment at 95° C.

### DETAILED DESCRIPTION OF ILLUSTRATIVE EMBODIMENTS

### Abbreviations and Acronyms

EC (or E.C.): Enzyme Commission number (EC number);

FPa: Fungal Protease 500,000 (Genencor—A Danisco Division);

FPb (or FPC): Fungal Protease Concentrate (Genencor—A Danisco Division);

HCl: Hydrochloric acid;

IP₃: Inositol triphosphate;

o/w: oil in water emulsion (also sometimes referred to herein as “cream”);

PIP₂: Phosphotidylinositol-bisphosphate;

PL1: lysophospholipase, (G-ZYME® G999);

PL2: phospholipase A, (LysoMax®)

P6L: Protex 6L;

pI: Isoelectric point;

w/w: weight by weight (usually expressed as a percentage, as in “% w/w” or % (w/w)).

### DEFINITIONS

As used herein an “emulsion” comprises an at least transiently stable system comprising a physical mixture of at least two materials, not completely miscible with, or soluble in, each other. Preferred emulsions, as used herein, do not readily separate when they are allowed to stand undisturbed, and can remain mixed for considerable lengths of time. They preferably will remain stable for extended periods of time such as greater than about 1, 2, 4, 8 12, or 24 hours, or even longer.

While emulsion systems can comprise solids, liquids, and gases, preferably, the emulsions for use herein comprise at least a lipid phase and an aqueous phase, both of which are in the liquid state. One phase in an emulsion is continuous, while the other phase is dispersed in and thus, discontinuous with, the other. For example, an “oil-in-water” emulsion has a discontinuous phase of oil dispersed in a continuous aqueous phase, whereas a “water-in-oil” emulsion has a discontinuous aqueous phase dispersed in a continuous lipid or oil phase. In a practical sense, the discontinuous phase consists of small droplets dispersed in and contained within the other phase. The discontinuous phase is thus also sometimes called the “internal” phase, and by analogy, the continuous phase is sometimes called the “external” phase. Under certain circumstances an emulsion can invert, i.e. the continuous and discontinuous phases may change roles, for example, an oil-in-water emulsion becomes a water-in-oil emulsion or vice versa.

It is to be understood that the use of the terms “oil-in-water” and “water-in-oil” herein are merely descriptive to help the reader understand which of the phases are continuous and which are dispersed in a given emulsion system; they do not limit the emulsion literally to oil and water. The “water” or aqueous phase may contain one or more solutes, such as salts, and any number of other soluble, or partially soluble compounds. Similarly, the “oil” or lipidic phase may contain a wide variety of lipids or lipid-soluble compounds.

Some compounds have the ability to stabilize emulsions, for example, by altering the surface tension and/or interfacial tension between the continuous and discontinuous phases, by preventing the dispersed discontinuous phase “droplets” from aggregating or coalescing, or even by increasing the viscosity of the continuous phase. Molecules that have both charged (e.g. ionic) and uncharged (nonionic) portions often serve as emulsion stabilizers. Because they are amphiphilic molecules, possessing both lipophilic and hydrophilic properties, they tend to concentrate at the interfaces between the two phases and control surface tension. Examples of stabilizers include many proteins, surfactants, glycerol, monoglycerides, diglycerides, and phospholipids. One group of phospholipids is known as lecithins. Widely used as natural emulsifiers, lecithins encompass several components, including phosphatidylcholine, phosphatidylethanolamine, phosphatidylinositol, and/or phosphatidic acid. Many such stabilizers are naturally present in plants and more specifically in lipid-containing and lipid-storing tissues of plants, such as oilseeds and fractions thereof. In particularly preferred embodiments exemplified herein, the oilseed is soybean. Soybeans are known to contain significant amounts of phospholipids (e.g. lecithins) and proteins that are capable of stabilizing emulsions.

As used herein “oil” denotes any of a group of fats (or lipids) that remain liquid at room temperature. “Plant oil” as used herein denotes any such oils that are obtained from any tissue of a plant. Plant oils are also referred to herein alternatively as “plant-derived oils”. In certain embodiments plant oils are edible, while in other embodiments they are not necessarily edible. Some oils may be appropriately and safely used for external application to an animal such as a human, while other oils may be completely inedible, and also not safe for external use on an animal. Such oils may nonetheless be valuable for use industrial process, as lubricants, cleaning or polishing products, or simply as feedstock for making other compositions or products requiring a lipid as a raw material. For example, some lipids are useful as fuels or fuel supplements. There is presently significant interest in biological or renewable sources of fuels, such as combustible fuels, for example lipids for use in biodiesel.

“Oilseed” as used herein refers to any oil-containing seed, nut, kernel, or the like produced by a plant. All such plants, as well as their seeds, nuts, or kernels are contemplated for use herein. For example, the National Sustainable Agriculture Information Service lists the following as sources of oil for food, specialty, or industrial uses: almonds, apricot kernels, avocado, beech nut, bilberry, black currant, borage, brazil nut, calendula, caraway seed, cashew nut, castor seed, citrus seed, clove, cocoa, coffee, copra (dried coconut), coriander, corn seed, cotton seed, elderberry, evening primrose, grape seed, groundnut, hazelnut, hemp seed, jojoba, linseed, macadamia nut, mace, melon seed, mustard seed, neem seed, niger seed, nutmeg, palm kernel, passion fruit, pecan, pistachio, poppy seed, pumpkin seed, rape seed, raspberry seed, red pepper, rose hip, rubber seed, safflower seed, sea buckthorn, sesame seed, soybean, spurge, stinging nettle, sunflower seed, tropho plant, tomato seed, or walnut. Also useful herein are various oilseed and related plants whose oil content is of interest for use as fuel, such as “eco-fuel”, biodiesel or the like. Such plants include but are not limited to jatropha (e.g. Jatropha curcas, J. mahafalensis, and cultivars thereof); Elaeis guineensis (e.g. Oil palm), Aleurites fordii (tung oil tree or wood oil tree), Ricinus communis (castor bean tree), Copaifera langsdorfii (diesel tree), and Pongammia pinnata (Honge oil tree, or Pongam tree, and cultivars thereof).

An “oil-containing fraction” or “lipid-containing fraction” as used herein refers to the oilseed or some portion or part thereof, however obtained. In preferred embodiments, the oil-containing fraction will comprise all, or at least a majority of, the oil (or fat or lipid content) of the oilseed. In other embodiments, a prior processing step may remove at least some, or even a majority of the oil from the oilseed prior to obtaining the fraction. Thus, for example, in certain embodiments using soybean as source of oilseed, the “fraction” comprises soy flour or soy flakes. Preferably, the flour or flakes obtained are “full-fat” as that term is understood in the art. However, that does not preclude the use of the processes provided herein with, for example, partially defatted fractions, such as soy flour or soy flakes. Economic factors may provide commercial motivation to use fractions containing at least a majority of the fat, however there are no known technical barriers to using the disclosed processes fractions comprising less than a majority of the fat of the whole oilseed.

For certain embodiments, the oil-containing fraction is from a major source of food or industrial oil. Presently, soybean, corn seed, cotton seed, and rape seed, as well as sunflower seed, safflower, flax seed, and peanut are preferred as sources of food oil.

As used herein, an “aqueous solvent” comprises at least water. Aqueous solvents typically comprise other components such as salts, buffering compounds, small molecules, and more. Any number and concentration of additional components may be present provided the aqueous solvent is substantially a homogeneous solution or suspension in water. This aqueous solvent, with the additional components as a substantially homogenous solution or suspension, is sometimes referred to herein as an “aqueous extractant” for convenience and to distinguish it from the solvent per se, e.g. water. The two terms are synonymous as defined herein. Preferably all components present are in true solution in the aqueous extractant or aqueous solvent. An aqueous solvent is distinguished from an organic solvent in that rather than water as the solvent, the term “organic solvent” refers to most other solvents that are organic compounds and contain carbon atoms. Organic solvents are more volatile and potentially explosive than aqueous solvents. Typically, organic solvent extraction of oils refers to any process used to remove an oil from oilseeds through direct contact with an organic solvent such as n-hexane or other hexanes.

“Microbial” as used herein to refer to a source of enzyme activities, for example, includes all single-celled and simple multicellular life forms, including but not limited to bacterial, fungal, algal (micro and macro), yeast, protozoan and the like. Preferred sources are often bacterial or fungal, especially those organisms which have historically or frequently been the source of enzymes for food processing that are generally recognized as safe or the like.

“Enzyme activity” as used herein refers to a chemical reaction catalyzed by one or more catalytic proteins (enzymes). A particular enzyme, or enzyme preparation may provide one or more enzyme activities. For example, a pure enzyme may be capable of catalyzing more than one enzymatic reaction (i.e. conversion of a substrate to product(s)) and thus, can be said to have more than one “enzyme activity”. The ability to convert a particular substrate to a corresponding product or products is, for purposes herein, an “enzyme activity” without regard to the number of proteins or their purity. In many cases, commercial enzyme preparations are not biochemically “pure” enzyme(s). Such preparations may provide a variety of enzyme activities in one or more physical enzymes. Some commercial enzyme preparations are designed to provide more than one enzyme activity, to allow a broader range of applications for the enzyme preparation. A pure enzyme having a single enzyme activity may not provide enough functionality to be useful under processing conditions, or in a complex system, such as a food matrix. Accordingly, for purposes herein, an “enzyme activity” is not necessarily synonymous with an enzyme. Each enzyme activity may be catalyzed by one or more enzymes, and a given enzyme or enzyme preparation may have one or more such activities.

“Diesel fuel extender” as used herein comprises any composition which can be used as a replacement or substitute for, or in lieu of diesel or any petrochemical-derived fuel, or to extend, dilute, or improve the efficiency of the use of such diesel or other petrochemical-derived fuel. As used herein “diesel fuel extender” may be a partial or complete fuel substitute, a fuel additive, or an alternative fuel source. “Bio-diesel” and “eco-fuel” are terms used in the art to denote example of such fuels which offer advantages including deriving from renewable resources, or even waste plant-based oil. As used herein, “diesel fuel extenders” may be compatible for use in standard fuel burning systems, for example standard diesel engines (such as diesel trucks, buses, or automobiles), or may only be suitable for use in fuel burning systems that are specially adapted for burning such fuels. Such fuel may be used for any purpose normally used for petrochemical based fuels, e.g. power generation, heat production, motor fuel, and the like. A plant oil obtained or prepared in accordance with the methods or processes provided may have an initial use for example in the food industry, and a secondary use as a fuel in accordance with the foregoing. Thus, it is contemplated that certain plant oils prepared by or recovered using the instant disclosure will be useful for one, two, or even more purposes prior to it ultimate use as a fuel.

In a first of several aspects, methods or processes are provided for destabilizing an emulsion comprising an oil phase and an aqueous phase. The emulsion is derived from, or produced in, an aqueous solvent extraction of a lipid from an oilseed. The emulsion is contacted with at least one enzyme activity including at least a phospholipase or a protease, under conditions allowing enzyme activity, for a time sufficient to destabilize the emulsion.

In preferred embodiments, the water phase is a continuous phase and the oil phase is a discontinuous phase, i.e. the emulsion is an oil-in-water emulsion.

In certain embodiments, the processes further include a separating step to separate the oil phase from the aqueous phase. Processes for separating oil from aqueous phases are known in the art. Frequently, they involve gravity or more preferably forces in excess of gravity, for example forces applied through physical means such as centrifugation. In one embodiment, the emulsions are centrifuged in a batch-wise process. In various embodiments, the conditions for separating may include adjusting the emulsion after enzyme treatment to a preferred temperature, for example by heating. In another embodiment, continuous centrifugation is preferred for separating the oil from the aqueous phase. Continuous processes may be preferred to larger-scale operations and are well-suited to handling material by the vessel-full, for example, from silos, tanks, vats, or the like, or even in connected series of such vessels.

Preferably, the processes provided herein improve the yield of oil from the emulsion. The skilled artisan will appreciate how to monitor yield on a variety of bases. Generally, the yield comparisons are made by comparing, for example, the yield of oil (for example on a % of theoretical maximum yield based on the oil content of the untreated emulsion) using the processes disclosed herein to the yield of an aqueous extraction that does not use the step of contacting the emulsion with the enzyme activity. Other bases of yield may be used, for example, an improvement of oil recovery using the processes disclosed herein versus a “control” process.

In preferred embodiments, the emulsion is contacted with at least one enzyme activity comprising at least a phospholipase activity or a protease activity. Enzymes combinations or mixtures that include one or more of either or both of the foregoing types of activities are also suitable. While such enzyme activities from any source such as animal, plant, or microbial, are contemplated for use herein, the enzyme activity preferably comprises a phospholipase activity from a mammalian pancreas, Streptomyces violaceoruber, Aspergillus oryzae, or Aspergillus niger. In one embodiment, the phospholipase activity is from porcine pancreas.

Various phospholipase activities have proven useful for destabilizing the emulsion in accordance with the processes disclosed herein. Phospholipases for purposes herein include, but are not limited to, phospholipases A (including A1 and A2), B (also sometimes referred to as lysophospholipase), C, and D. Phospholipases are a class of enzymes that hydrolyze phospholipids, such as phosphatidylcholine or phosphatidylethanolamine. Within the phospholipase class of enzymes are five major subclasses, A1, A2, B, C, and D phospholipases.

A1 phospholipases (E.C. 3.1.1.32) preferentially hydrolyze the sn1 ester bonds of phospholipids, such as phosphatidylcholine or phosphatidylethanolamine, to yield 1-lysophospholipids plus carboxylic acids. Typically, A1 phospholipases require calcium as a cofactor. A1 phospholipases generally exhibit broader specificity than A2 phospholipases.

A2 phospholipases (E.C. 3.1.1.4) preferentially hydrolyze the sn2 ester bonds of phospholipids, such as phosphatidylcholine or phosphatidylethanolamine, to yield 2-lysophospholipids plus carboxylic acids. In addition to phospholipids, A2 phospholipases show some specificity for hydrolysis of choline derivatives and phosphatides. Typically, A2 phospholipases require calcium as a cofactor.

B phospholipases (E.C. 3.1.1.5) are also known as lysophospholipases. They preferentially hydrolyze the sn1 ester bonds of 2-lysophospholipids to yield glycerophosphatides plus carboxylic acids. B phospholipases will also hydrolyze the sn2 ester bonds of 1-lysophospholipids.

C phospholipases (E.C. 3.1.4.3) preferentially hydrolyze the phosphate bonds of phospholipids, such as phosphatidylcholine or phosphatidylethanolamine, to yield the corresponding diacylglycerols and choline phosphates. In addition to hydrolysis of phospholipids, C phospholipases will also act on lysophospholipids.

D phospholipases (E.C. 3.1.4.4) preferentially hydrolyze the phosphate bond of phospholipids such as phosphatidylcholine or phosphatidylethanolamine to yield the corresponding phosphatidic acids and choline. In addition to hydrolysis of phospholipids, D phospholipases will also act on lysophospholipids.

Phospholipases can be used individually or in combination or mixtures of one or more activities of the same or different E.C. classifications, and from the same or different sources. Crude or partially purified enzyme preparations containing one or more phospholipase activities are suitable for use in some embodiments herein. Commercial sources of phospholipases are also suitable for use herein. For example, Genencor—A Danisco Division (Rochester, N.Y.) offers LysoMax® and G-ZYME® G999 phospholipases, from bacterial and fungal sources, respectively. Phospholipase C is available commercially, for example, from Sigma (St. Louis, Mo.).

In other embodiments, the enzyme activity comprises a protease activity, such as a protease activity from Bacillus amyloliquifaciens. In yet other embodiments the enzyme activity comprises one or more protease activities from a plant, animal or microbial source. Presently preferred protease activities derive from microbial sources including B. subtilis, B. lichenformis, A. niger or A. oryzae, in addition to B. amyloliquifaciens described above. As with phospholipases, combinations or mixtures, whether purified, partially purified, or crude, comprising one or more protease activities of any E.C. classification and from any source are suitable for use herein. In one embodiment, the protease activity comprises an endopeptidase. Metalloproteases, whether exo- or endo-proteases, are suitable for use with the processes and compositions disclosed. Metallo-endoproteases are preferred in some embodiments. Fungal proteases are also suitable for use in certain embodiments. Many commercial sources of proteases are appropriate. Genencor—A Danisco Division (Rochester, N.Y.) offers Fungal Protease 500,000 and Fungal Protease Concentrate proteases, each of which comprises neutral to acid stable protease activity, and each of which is exemplified herein. They also offer Protex 6L protease, also exemplified herein, which comprises bacterial protease activity and prefers neutral to slightly alkaline conditions.

In another aspect, processes are provided for obtaining oil from an oilseed comprising the steps of:
(a) providing an oil-containing oilseed fraction;
(b) contacting the oil-containing oilseed fraction with an aqueous extractant to form an extracted oilseed fraction;
(c) separating the extracted oilseed fraction into an aqueous phase, an oil-in-water emulsion, and an insoluble phase;
(d) contacting the oil-in-water emulsion with at least one enzyme activity capable of destabilizing the emulsion under conditions permitting enzyme activity for a time sufficient to destabilize the emulsion; and
(e) separating the destabilized emulsion into an aqueous phase, an oil phase, and an insoluble phase;

thereby obtaining oil from the oilseed.

In one embodiment, the oil-containing oilseed fraction comprises cells and the process further comprises the step of disrupting the cells prior to contacting the oilseed fraction with the aqueous extractant. Presently preferred methods of disrupting cells include application of a mechanical force. Extrusion is a convenient method of disrupting cells in oil-containing seeds. Such use of extruders is known in the art, for example single-screw or twin-screw extruders are useful therefore. The skilled artisan can readily determine parameters for disrupting cells in oilseeds by measuring the oil recovery or yield, while varying the extrusion parameters such as speed, transit time, pressure, temperature, pH, and the like. Other methods of disrupting cells include chemical and or enzymatic treatment, in addition to additional mechanical methods such as pressing, rolling, abrading, sonication, and others.

In some presently preferred embodiments, the oil-in-water emulsion is contacted with enzyme activity comprising at least a phospholipase activity or a protease activity, for example from an animal, plant, or microbial source. Combinations or mixtures of one or more such enzymes activities are also expressly included for use herein. In one embodiment, the enzyme activity comprises a phospholipase activity from a mammalian pancreas (e.g. porcine, bovine, equine, ovine, or other), or from a microbial source whether bacterial, fungal, algal or the like, for example, from Streptomyces violaceoruber, Aspergillus oryzae, or Aspergillus niger. In a preferred embodiment the enzyme is available from an organism that is generally recognized as safe or useful with consumables such as human food and animal feed, and other consumer products such as cosmetics and skin care products.

In another embodiment, the enzyme activity comprises a protease activity, for example from a bacterial, fungal, or algal source. One presently preferred bacterial source of protease activity is Bacillus amyloliquifaciens. Other sources presently preferred for use herein are from microbial sources including for example, B. subtilis, B. lichenformis, A. niger, and A. oryzae. In certain embodiments, the protease activity comprises an endopeptidase, which can be a metalloprotease in various embodiments.

The process can include a centrifugation step for either or both of the separating steps. The skilled artisan will appreciate the use of centrifugation for separating phases of differing densities, such as the oil and aqueous phases present in the emulsion of the process provided.

In some embodiments, the pH is adjusted to between about 3.5 to about 5 prior to the separating step. Such adjustment may help to destabilize the emulsion in a variety of ways. Without limiting the process to any one theory of operation, a pH adjustment may help to precipitate one or more proteins, said proteins perhaps involved in stabilizing the emulsion. Alternatively, they may minimize charge difference on groups that slow or prevent the discontinuous phase from coalescing or aggregating. The pH adjustments may also work by allowing the enzymes to function better in the destabilization process—e.g. allowing the enzyme and substrate to more quickly associate, enhancing actual catalysis, or by helping the product to more quickly diffuse or leave the active site, thus driving the conversion of substrate to product.

In one embodiment, the aqueous extractant itself comprises one or more enzymes. Preferably the enzymes include one or more of a protease, a cellulase, a hemicellulase, a pectinase, a glucanase, a phospholipase, a lipase, a lecithinase, or an amylase. Such activities can aid in the recovery of oil and the breakdown of cellular material retaining said oil.

In some cases, at least one phospholipase activity or one protease activity in the aqueous extractant separates with the oil-in-water emulsion at the first separating step. The surviving or co-separating enzyme (phospholipase or protease) is sometimes referred to herein as carryover activity. The carryover activity is present with the oil-in-water emulsion and destabilizes that emulsion when provided with suitable conditions for activity, for an adequate time. Such a process of providing sufficient enzyme initially, then employing suitable process conditions to provide carryover activity in the later step can facilitate oil recovery by allowing the one-step addition to suffice for the entire process. The skilled artisan can readily determine whether sufficient enzyme activity has carried over into the oil-in-water emulsion.

In particular embodiments of this aspect, the oilseed fraction is from soybean, corn seed, rape seed, palm kernel, sunflower seed, safflower seed, coconut, peanut, cotton seed, sesame seed, flax seed, poppy seed, almond, hazelnut, walnut, evening primrose seed, grape seed, hemp seed, black currant seed, red raspberry seed, carrot seed, cumin seed, blueberry seed, cranberry seed, parsley seed, onion seed, pumpkin seed, apricot kernel, mustard seed, linseed, or castor seed. As discussed above, various species considered to be useful or potentially useful for biodiesel and similar fuel-related uses including, for example, jatropha, oil palm, tung oil or wood oil tree, castor bean tree, diesel tree, and honge oil tree and any cultivars of any of the foregoing are also contemplated for use herein.

In certain preferred embodiments, the oilseed fraction comprises a protein fraction that is useful as a food, a food ingredient, a food additive, or a food supplement. It is sometimes or even often the case that the protein fraction of the oilseed is more valuable than the oil fraction. One anticipated benefit of aqueous extraction processes is that they leave the extracted residue in a native or more functional state, e.g. the proteins are not denatured or retain greater functionality through contact with the aqueous solvent; this is valuable to the food processor. Whether for use as food or feed, or other use, the processes provided herein allow recovery of a protein fraction that has not been exposed to potentially dangerous or harmful organic solvents. As such the proteins are less likely to be denatured and may even have better consumer acceptance among certain groups of consumers.

In one embodiment, the oilseed fraction comprises soy flakes or soy flour. It is preferred that the oilseed fraction is “full-fat” flakes or “full-fat” flour. The skilled artisan will understand that “full-fat” is a term of the art in working with oilseed fractions such as from soy and corn. Full-fat is in one sense the opposite of defatted—wherein essentially all of the lipids are removed from, for example soy flakes, soy flour, or corn germ. The skilled artisan will appreciate that the process provided herein are compatible with not only full-fat fractions, but also with partially defatted fractions of oilseed—whether flakes, meal, germ, flour, or the like.

In certain embodiments, the conditions include adjusting the pH of the oil-in-water emulsion to between about 3.5 and about 5. There are a wide variety of methods for affecting pH adjustment—including but not limited to the addition of HCl, or other acids, including organic acids, or salts thereof. For food uses, such acids are preferably food ingredients, or generally recognized as safe for use in foods by the appropriate regulatory bodies.

In a preferred embodiment, the processes provided herein improve the yield of oil from the oilseed as compared to that of an aqueous solvent extraction that does not use the step of contacting the emulsion with the enzyme activity. In a more preferred embodiment, the oil recovery approaches that obtained with an organic solvent extraction.

In yet another aspect plant-derived oils are provided, said oils prepared by any of the processes described herein. In one embodiment, the oil is substantially free of proteins, phospholipids, or aqueous impurities. Also provided are food products comprising the oil.

In another aspect herein, compositions are provided that comprise at least one enzyme activity capable of de-stabilizing an oil-in water emulsion; and further comprise an oil-in-water emulsion obtained from an aqueous solvent extraction of an oil-containing oilseed fraction. Such compositions are intermediates in the processes provided. They are useful as starting material in the extraction of oil from an oil-in-water emulsion obtained from the aqueous solvent extraction of a oilseed. They may also be useful as a functional ingredient for adding directly to a food. These compositions can help provide a high-quality lipid component, while simultaneously providing high-quality functional protein and phospholipid content. Because the addition of the enzyme will tend to destabilize the emulsion, processors may find that this composition has utility for direct addition to a process. In other embodiments, preferably after destabilization of the emulsion, the composition is separated into at least a lipidic phase and an aqueous phase, wherein the enzyme activities separate with the aqueous phase. The lipidic and aqueous phases so separated are each useful as a component or ingredient in a food process.

In a preferred embodiment, the enzyme activity comprises at least a phospholipase or a protease. The enzyme activity as provided herein and above can comprise a phospholipase activity from an animal, plant, or microbial source, for example, mammalian pancreas, Streptomyces violaceoruber, Aspergillus oryzae, or Aspergillus niger. Phospholipase activity from porcine, equine, bovine, or ovine pancreas are currently preferred as animal sources. The enzyme activity can also or alternatively comprise a protease activity from an animal, plant or microbial source. For example, one protease is from a bacterial source such as Bacillus amyloliquifaciens, Bacillus subtilis, Bacillus lichenformis, or a fungal source such as Aspergillus niger, or Aspergillus oryzae. In certain embodiments the protease is an endopeptidase, such as a metalloprotease.

Also provided herein are plant-derived oils isolated from the foregoing compositions.

In another of its aspects, methods are provided for obtaining plant oil from an oilseed fraction. The methods comprise the steps of:
(a) providing a composition comprising at least one enzyme activity capable of de-stabilizing an oil-in water emulsion, and an oil-in-water emulsion obtained from an aqueous solvent extraction of an oil-containing oilseed fraction;
(b) providing conditions under which the enzyme activity de-stabilizes the oil-in-water emulsion; and
(c) separating the composition into at least an aqueous phase and a lipid phase, said lipid phase comprising the plant oil.

In certain embodiments, the conditions in step (b) include a pH that is about the isoelectric point of a protein present in the oil-in-water emulsion. A presently preferred pH is one that is between about 3.5 to about 5.

In various embodiments, the separating step further produces an insoluble portion, preferably which comprises a protein. As discussed above, aqueous solvent extraction processes can provide proteins that retain greater functionality than those produced by organic solvent extraction of oilseed plants. These proteins having the greater functionality have enhanced value, and often are more valuable than the oil derived from the oilseed.

Also provided herein are plant-derived oils comprising plant oil prepared by the foregoing method, or any other disclosed herein. Food products comprising a plant oil prepared by these methods are also provided, as are industrial feedstocks and consumer products comprising the plant oil so prepared.

Protein compositions prepared by the methods described herein are also provided, as are food products, consumer products, and industrial feedstock comprising a protein prepared by the methods or processes disclosed herein.

### EXAMPLES

### Example 1

### De-Emulsification of an Oil-in-Water Emulsion Obtained from Extruded and Enzyme Treated Full-Fat Soy Flakes

Screening of Enzymes: Protease and Phospholipase

Table 1-1 summarizes the enzymes that were tested on an oil-in-water emulsion at a concentration of 500 mg (2% w/w oil-in-water emulsion). About 1.2 kg of extruded full-fat soy flakes were used as the starting material. After adjusting the temperature to 50° C. with a temperature-controlled water bath equipped with a Lab-Stirrer LR 400C (Fisher Scientific) at a speed of 150 rpm., and adjusting the pH to 7.0 using 2N NaOH, Multifect Neutral® (Genencor—A Danisco Division) was added at 0.5% (w/w) (on the basis of soy flake weight). The temperature and pH of the reaction were maintained for 1 hr at the stated values. After the 1 hour incubation, the pH was raised to 8.0 for 15 min. Insoluble residue was removed by centrifugation in an SLA-3000 fixed-angle rotor (Sorvall RC5BPlus, Newtown, Conn.) at 3,000 g for 15 min. After centrifuging, the oil in water emulsion (including any separated oil) and aqueous fractions were collected into a funnel and stored at 4° C. overnight. The oil-in-water emulsion and free (separated) oil fraction were collected after layer separation and used for enzymatic de-emulsification. The enzyme reactions to demulsify the oil-in-water emulsion were incubated for 90 min. at an optimal temperature and pH for each enzyme. The enzymes were added either independently, in combination, or in sequence, according to the experimental design (see description in the footnote of Table 1-2).

TABLE 1-1
Summary and identification of enzymes tested.

| Enzyme | | Abbreviation | Incubation | |
|---|---|---|---|---|
| Activity | Source of Activity | Used | Temp (° C.) | pH |
| Phospholipase | LysoMax ® enzyme | PL2 | 40 | 8 |
| | G-ZYME ® G999 lyso-phospholipase | PL1 | 50 | 4.5 |
| Protease | Protex 6L | P6L | 50 | 8 |
| | Fungal Protease 500,000 | FPa | 50 | 4.5 |
| | Fungal Protease Concentrate | FPb | 50 | 8 |

Results:

The results are shown in Table 1-2. The concentration of enzyme in this first set of experiments was relatively high (500 mg, i.e. 2% weight by weight). Two enzymes were selected to determine impact of enzyme concentration on destabilization of the oil-in-water emulsion. Fungal Protease Concentrate (FPC) and LysoMax® enzyme, both working at pH 8, were selected.

When enzyme was added independently, substantially all of the oil was released (Yield of recovered oil equals ˜100%) regardless of the nature of enzyme. Because treatment with individual protease or phospholipases resulted in nearly 100% oil release, no conclusion can be drawn on the advantages of a combination of enzymes. Surprisingly, however, the lysophospholipase (PL1) worked as well as the phospholipase A2 (PL2). This was not expected because it was considered that the activity of PL1 converting the substrates of PL1 into the corresponding products might not destabilize the emulsion as much as the corresponding conversion of substrate to product by PL2. By another view, the potential substrates for PL1 were not deemed to be as responsible for the stability of the emulsion as were the substrates for PL2.

TABLE 1-2
Effects of enzyme on de-emulsification of oil-in-water emulsion.

| | | Percentage of Oil in |
|---|---|---|
| | Yield of Recovered | Residual Emulsion after |
| Enzyme (s) | Oil (%) | Oil Recovery (%) |
| Single Enzyme | | |
| PL2 | 100.7 | 3.0 |
| PL1 | 97.7 | 4.8 |
| P6L | 104.2 | 7.7 |
| FPa | 87.7 | 4.8 |
| FPb | 95.6 | 7.2 |
| Combination of Enzymes | | |
| Enzymes added together | | |
| PL2 + PL1* | 102.5 | 3.1 |
| PL2 + PFb* | 105.0 | 2.5 |
| PL2 + P6L* | | |
| Enzymes added | 96.5 | 6.2 |
| sequentially | | |
| PL2 − PL1 | 106.1 | 3.7 |

Note:
About 20 g of the oil-in-water emulsion was used for each test. Five hundred (500) mg of enzyme was added.
*the reaction temperature was 40° C. For abbreviations, see Table 1-1.
Note for results:
(A) Yield of recovered oil (%) is calculated as the ratio of oil (g) recovered after enzymatic incubation to the oil (g) in the original oil-in-water emulsion, expressed as a percentage; (B) Oil in residual emulsion after oil recovery (%) is calculated as the ratio of oil left in residual emulsion to the oil in the original oil-in-water emulsion, expressed as a percentage.

### Example 2

### Effect of Enzyme Concentration on Yield of Free Oil

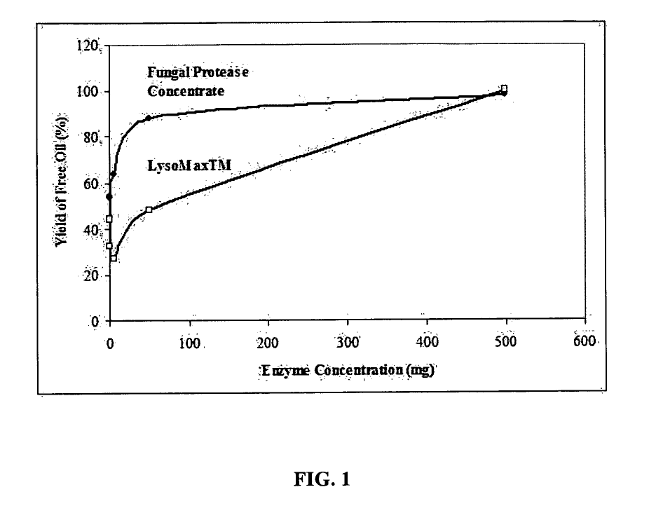

Enzyme (0.5, 5, 50, or 500 mg each) was added to 20 g samples of the oil-in-water emulsion. The amount of enzyme corresponds to a percentage of 0.002, 0.02, 0.2 and 2 (w/w) %, respectively. The results are shown graphically in FIG. 1. FIG. 1 shows the yield curves as a function of concentration for each of the enzymes, Fungal Protein Concentrate and LysoMax®, respectively. Each data point provided is the average of two independent experiments.

A yield of recovered oil of 44% was obtained for the control, i.e. oil-in-water emulsion without added enzyme activity. At 0.2% FPC added, 88% of the oil was recovered. In contrast, only 48% of the oil was recovered with the same concentration of LysoMax® enzyme. Thus, although either of the protease or the phospholipase activities were capable of improving the yield as the concentration of the enzyme was increased, at lower concentrations, Fungal Protease Concentrate was more effective in maximizing recovery of oil than LysoMax® enzyme.

### Example 3

### Effect of pH on De-Emulsification of Oil-in-Water Emulsion from Soybeans

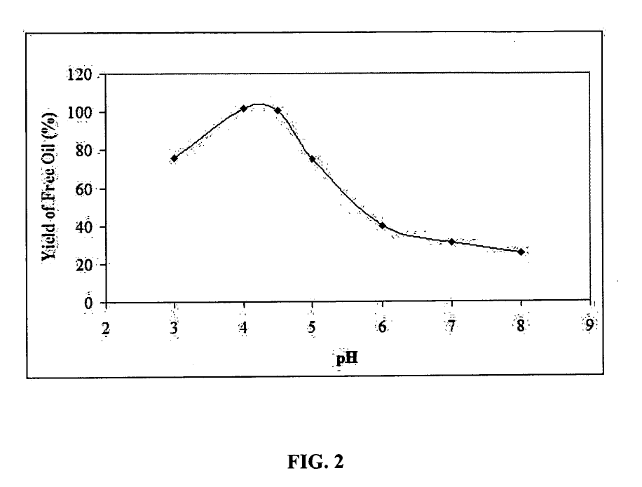

The pH of the emulsion was adjusted to the desired point with hydrochloric acid. The emulsion was stirred for 10 min at 50° C. before centrifuging, to separate and recover the oil. As can been seen from the graph provided in FIG. 2, there was a direct relationship between the pH value and the yield of oil recovered. The highest yields were obtained between about pH 4 and about 4.5, a range which corresponds to the pI of soy proteins. A pH of about 4.2-4.3 appeared to be an optimal condition for oil recovery from the emulsion. Recoveries of oil at pH's above about 6 were substantially less (about 40% or less) than those obtained at pH's below about 5.5 (about 50% or more). It was observed that the response curve was reminiscent of those obtained for the solubility of soy protein as a function of pH.

### Example 4

### Extraction of Full-Fat Soy Flakes with Extrusion and Aqueous Extraction

Conditions for extrusion of full-fat soy flakes were optimized using a twin-screw extruder (American Leistritz). It was determined that extrusion at 100° C., at a screw speed of 100 rpm and the moisture content at about 14-15% provided preferred results.

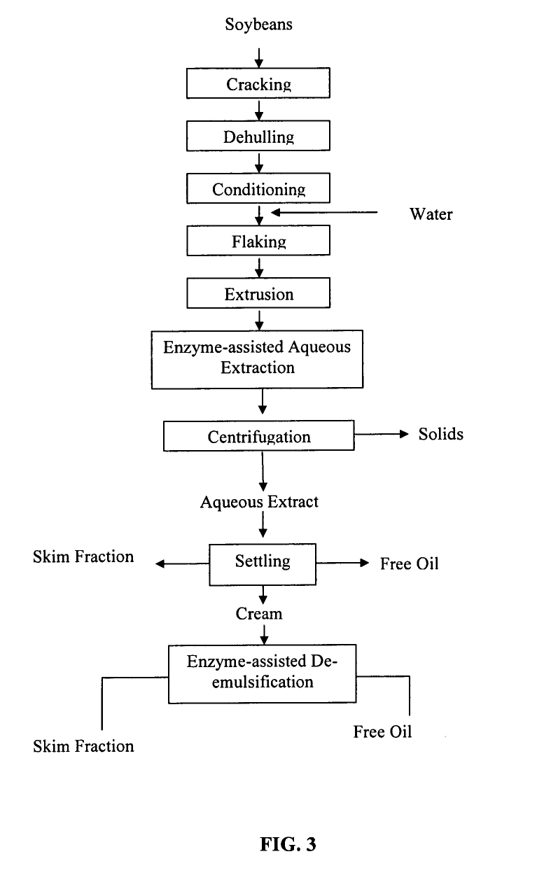

Full-fat soy flakes were extruded directly into water. The aqueous soy flake mixture was treated with an enzymatic step by adding enzyme (Multifect Neutral, Genencor—A Danisco Division) at 0.5% w/w on a dry weight solids basis. The mixture was incubated at 50° C. for 1 hr with agitation. After the incubation the mixture was separated by centrifugation at about 3000×g to yield an insoluble fraction and aqueous supernatant. The aqueous supernatant comprised oil (“free oil”), an oil-in-water emulsion, and a aqueous fraction. Thus the totality of the oil was either free or in the emulsion. The free oil and the aqueous fraction were separated by gravity settling overnight in the cold (less than about 10° C.). The heavier aqueous fraction was first removed, and then the oil and emulsion were collected together. FIG. 3 is a flow chart showing the process as used. The skilled artisan will appreciate that the process can be varied in certain steps in keeping with the spirit of the methods and compositions provided.

The emulsion was then subjected to an enzymatic de-emulsification process. The enzymes selected for this step were phospholipases. Emulsion samples were treated with either of two phospholipase activities under the conditions provided below:

Enzyme Treatment 1: Phospholipase C: Enzyme was obtained from Sigma (Product Number P-7633-500UN, 10-50 units/mg, Sigma, St. Louis, Mo.). About 15 mg of enzyme were added to about 18-20 g of oil plus emulsion mixture as described above. Agitation was provided during the 90 min incubation at 37° C. and pH 7.

Enzyme Treatment 2: Phospholipases: LysoMax® and G-ZYME G999 enzymes (Genencor—A Danisco Division) were used combined in a 1:1 ratio. About 500 mg of each of the enzymes were added to about 18-20 g of the emulsion and oil mixture. Samples were agitated during the 90 min incubation at 50° C. and at a pH of about 5.

After the incubation, the destabilized oil plus oil-in-water emulsion mixtures were separated by centrifuging at about 1000×g. The oil was collected. The results were expressed as recovered oil, which was the percentage of the total oil in the extruded flakes. The total oil in the extruded flakes was measured based on the Mojonnier acid hydrolysis procedure (AOAC Method 922.06, the entirety of which is incorporated herein by reference). See AOAC, Official Methods of Analysis, 15th edition, Association of Official Analytical Chemists, Washington, D.C.

Results: The average oil recovery after the extrusion plus aqueous extraction followed by the de-emulsification was 73% for Enzyme Treatment 1 versus 68% for Enzyme Treatment 2. These were not statistically different at the p<0.05 level. Statistical calculations were determined using the General Linear Model, PROC GLM, SAS statistical software (version 8.2, SAS Institute, Inc., Cary, N.C.). Typical oil recoveries approach about 97% of the total oil when using organic extraction methods, such as with hexane. Aqueous extraction procedures typically extract oil in emulsified form. When extrusion or other pretreatment is used prior to aqueous extraction, about 10-15% of total oil is recovered as free oil not in the emulsified form.

A comparison was also made of the enzymatic de-emulsification step with other physical or mechanical methods known to break emulsions, for example heating, and repeated freeze-thaw cycles. These processes, as the skilled artisan will appreciate can not only be more difficult to implement than an enzymatic step, but they can be energy intensive. The treatments actually tested were as follows:

Control treatment: an aqueous extract from extruded soy flakes was centrifuged and the centrifuge supernatant was further fractionated using a separatory funnel to yield the residual o/w emulsion (“cream” fraction), the soluble fraction (“skim” fraction), and free oil.

Phospholipase C treatment: an aqueous extract from soy flakes was centrifuged. The supernatant fraction was then treated with 0.075% (w/w) phospholipase C at 37° C. and pH 7 for 90 minutes. The phospholipase C-treated supernatant was further fractionated via separatory funnel to yield the residual cream, skim, and free oil fractions (as described above).

Genencor Enzyme Cocktail treatment: an aqueous extract from soy flakes was centrifuged. The supernatant was treated with 2.5% (w/w) LysoMax® plus 2.5% (w/w) G-ZYME G999 at 50° C. and pH 5 for 90 minutes. The enzyme-treated supernatant was further fractionated via separatory funnel to yield the residual cream, skim, and free oil fractions.

Freeze-Thaw treatment: an aqueous extract from extruded soy flakes was centrifuged and the centrifuge supernatant was subjected to 3 freeze-thaw cycles. Then the treated supernatant was further fractionated via a separatory funnel to yield the residual cream, skim, and free oil fractions.

95° C. Heat treatment: an aqueous extract from extruded soy flakes was centrifuged. The centrifuge supernatant was heated to 95° C. and held at this temperature for 30 minutes, and then cooled to 25° C. The treated supernatant was further fractionated via a separatory funnel to yield the residual cream, skim, and free oil fractions.

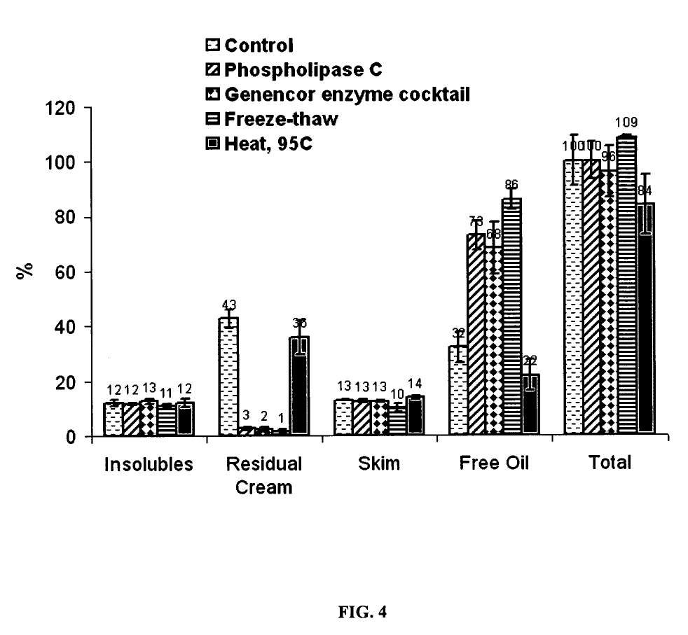

The results of the foregoing comparison experiment are shown in FIG. 4.

Various publications, including patents, published applications, technical articles and scholarly articles are cited throughout the specification. Each of these cited publications is incorporated by reference herein, in its entirety for all purposes.

What is claimed:

1. A process for destabilizing an emulsion comprising an oil phase and an aqueous phase, said emulsion produced in an aqueous solvent extraction of a lipid from an oilseed, the process comprising the step of contacting the emulsion with at least one enzyme activity including at least a phospholipase or a protease, under conditions allowing activity of at least the phospholipase or the protease for a time sufficient to destabilize said emulsion.

2. The process of claim 1, wherein the water phase is a continuous phase and the oil phase is a discontinuous phase.

3. The process of claim 1, further comprising separating the oil phase from the aqueous phase.

4. The process of claim 3, wherein the separating step comprises centrifugation.

5. The process of claim 1 which improves the yield of oil from the oilseed as compared to that of an aqueous extraction that does not use the step of contacting the emulsion with the enzyme activity.

6. The process of claim 1, wherein the emulsion is contacted with at least one enzyme activity comprising at least a phospholipase activity or a protease activity, or a combination thereof.

7. The process of claim 6, wherein the enzyme activity comprises a phospholipase activity from a mammalian pancreas, Streptomyces violaceoruber, Aspergillus oryzae, or Aspergillus niger.

8. The process of claim 7, wherein the enzyme activity comprises a phospholipase activity from porcine pancreas.

9. The process of claim 6, wherein the enzyme activity comprises a protease activity from Bacillus amyloliquifaciens, Bacillus subtilis, Bacillus lichenformis, Aspergillus niger, or Aspergillus oryzae.

10. The process of claim 6, wherein the protease activity comprises an endopeptidase.

11. The process of claim 10, wherein the endopeptidase is a metalloprotease.

12. The process of claim 3, wherein the pH is adjusted to between about 3.5 to about 5 prior to the separating step.

13. A process for obtaining oil from an oilseed comprising the steps of:
(a) providing an oil-containing oilseed fraction;
(b) contacting the oil-containing oilseed fraction with an aqueous extractant to form an extracted oilseed fraction;
(c) separating the extracted oilseed fraction into an aqueous phase, an oil-in-water emulsion, and an insoluble phase;
(d) contacting the oil-in-water emulsion with at least one enzyme activity under conditions permitting enzyme activity for a time sufficient to destabilize the emulsion; and
(e) separating the destabilized emulsion into an aqueous phase, an oil phase, and an insoluble phase;
thereby obtaining oil from the oilseed.

14. The process of claim 13, wherein the oil-containing oilseed fraction comprises cells and the process further comprises the step of disrupting the cells prior to contacting the oilseed fraction with the aqueous extractant.

15. The process of claim 14, wherein the disrupting step comprises application of a mechanical force.

16. The process of claim 14, wherein the disrupting step comprises extrusion.

17. The process of claim 13, wherein the oil-in-water emulsion is contacted with enzyme activity comprising at least a phospholipase activity or a protease activity, or a combination thereof.

18. The process of claim 17, wherein the enzyme activity comprises a phospholipase activity from a mammalian pancreas, Streptomyces violaceoruber, Aspergillus oryzae, or Aspergillus niger.

19. The process of claim 18, wherein the enzyme activity comprises a phospholipase activity from porcine pancreas.

20. The process of claim 17, wherein the enzyme activity comprises a protease activity from Bacillus amyloliquifaciens, Bacillus subtilis, Bacillus lichenformis, Aspergillus niger, or Aspergillus oryzae.

21. The process of claim 20, wherein the protease activity comprises a endopeptidase.

22. The process of claim 21, wherein the endopeptidase is a metalloprotease.

23. The process of claim 13, wherein either or both of the separating steps comprise centrifugation.

24. The process of claim 23, wherein the pH is adjusted to between about 3.5 to about 5 prior to the separating step.

25. The process of claim 13, wherein the aqueous extractant comprises one or more enzymes.

26. The process of claim 25, wherein the enzymes include one or more of a protease, a cellulase, a hemicellulase, a pectinase, a glucanase, a phospholipase, a lipase, a lecithinase, or an amylase.

27. The process of claim 26, wherein the enzymes comprise at least a phospholipase activity or a protease activity, and at least a portion of said enzyme activity in the aqueous extractant separates into the oil-in-water emulsion and destabilizes the oil-in-water emulsion.

28. The process of claim 13, wherein the oilseed fraction is from soy bean, corn seed, rape seed, palm kernel, sunflower seed, safflower seed, coconut, peanut, cotton seed, sesame seed, flax seed, poppy seed, almond, hazelnut, walnut, evening primrose seed, grape seed, hemp seed, black currant seed, red raspberry seed, carrot seed, cumin seed, blueberry seed, cranberry seed, parsley seed, onion seed, pumpkin seed, apricot kernel, mustard seed, linseed, castor seed, or jatropha.

29. The process of claim 28, wherein the oilseed fraction comprises a protein fraction that is useful as a food, a food ingredient, a food additive, or a food supplement.

30. The process of claim 28, wherein the oilseed fraction comprises soy flakes or soy flour.

31. The process of claim 30, wherein oilseed fraction is full-fat flakes or full-fat flour.

32. The process of claim 13, wherein the conditions include adjusting the pH of the emulsion to between about 3.5 and about 5.

33. The process of claim 13 which improves the yield of oil from the oilseed as compared to that of an aqueous solvent extraction that does not use the step of contacting the emulsion with the enzyme activity.

34. A plant-derived oil prepared by the process of claim 1.

35. A food product comprising the oil of claim 34.

36. The oil of claim 34 that is substantially free of proteins, phospholipids, or aqueous impurities.

37. A composition comprising:
at least one enzyme activity capable of de-stabilizing an oil-in water emulsion; and
an oil-in-water emulsion obtained from an aqueous solvent extraction of an oil-containing oilseed fraction.

38. The composition of claim 37, wherein the enzyme activity comprises at least a phospholipase or a protease, or a combination thereof.

39. The composition of claim 38, wherein the enzyme activity comprises a phospholipase activity from a mammalian pancreas, Streptomyces violaceoruber, Aspergillus oryzae, or Aspergillus niger.

40. The composition of claim 39, wherein the enzyme activity comprises a phospholipase activity from porcine pancreas.

41. The composition of claim 38, wherein the enzyme activity comprises a protease activity from Bacillus amyloliquifaciens, Bacillus subtilis, Bacillus lichenformis, Aspergillus niger, or Aspergillus oryzae.

42. The composition of claim 41, wherein the protease activity comprises an endopeptidase.

43. The composition of claim 42, wherein the endopeptidase is a metalloprotease.

44. A plant-derived oil isolated from the composition of claim 37.

45. A method for obtaining plant oil from an oilseed fraction comprising the steps of:
(a) providing a composition comprising at least one enzyme activity capable of de-stabilizing an oil-in water emulsion, and an oil-in-water emulsion obtained from an aqueous solvent extraction of an oil-containing oilseed fraction;
(b) providing conditions under which the enzyme activity de-stabilizes the oil-in-water emulsion; and
(c) separating the composition into at least an aqueous phase and a lipid phase, said lipid phase comprising the plant oil.

46. The method of claim 45, wherein the conditions in step (b) include a pH that is about the isoelectric point of a protein present in the oil-in-water emulsion.

47. The method of claim 46, wherein the pH is between about 3.5 to about 5.

48. The method of claim 46, wherein the separating step further produces an insoluble portion.

49. The method of claim 48, wherein the insoluble portion comprises a protein.

50. A plant-derived oil comprising plant oil prepared by the method of claim 45.

51. A food product comprising plant oil prepared by the method of claim 45.

52. An industrial feedstock comprising plant oil prepared by the method of claim 45.

53. A consumer product comprising plant oil prepared by the method of claim 45.

54. A protein composition prepared by the method of claim 49.

55. A food product comprising protein prepared by the method of claim 49.

56. A consumer product comprising protein prepared by the method of claim 49.

57. An industrial feedstock comprising protein prepared by the method of claim 49.

58. A diesel fuel extender comprising a plant oil prepared by the method of claim 1 or claim 45.
